(19) United States
(12) Patent Application Publication
(10) Pub. No.: US 2012/0309090 A1
(43) Pub. Date: Dec. 6, 2012

(54) SELF CONTAINED SOLID PHASE PHOTOBIOREACTOR

(75) Inventors: John Aikens, La Grange Park, IL (US); Robert J. Turner, Aurora, IL (US); Denise L. Holzle, Bolingbrook, IL (US)

(73) Assignee: PROTERRO, INC., Princeton, NJ (US)

(21) Appl. No.: 13/487,070

(22) Filed: Jun. 1, 2012

Related U.S. Application Data
(60) Provisional application No. 61/493,139, filed on Jun. 3, 2011.

(51) Int. Cl. C12M 1/42; C12M 1/12; C12N 1/12; C12N 1/14; C12N 1/10; C12N 5/04; C12M 1/00; C12N 1/20

(52) U.S. Cl. 435/420; 435/289.1; 435/297.1; 435/292.1; 435/252.1; 435/254.1; 435/258.1; 435/257.1

(57) ABSTRACT

A compressible bioreactor for cultivating photosynthetic microorganisms, the bioreactor comprising a feeder trough, a collection trough, a growth fabric, and a barrier layer, where the bioreactor has a compressed mode and an extended mode, the growth fabric is coupled to the feeder trough and the collection trough, the growth fabric is substantially extended in the extended mode of the bioreactor, the growth fabric is substantially compressed in the compressed mode of the bioreactor and the barrier layer is coupled to the feeder base and the collection base that encases the growth fabric in a substantially airtight environment

### CROSS-REFERENCE TO RELATED APPLICATIONS

This application claims the benefit of U.S. Provisional Application Ser. No. 61/493,139, filed on Jun. 3, 2011, which is incorporated herein by reference in its entirety.

### BACKGROUND OF THE INVENTION

Photobioreactors cultivate organisms in a liquid based culture medium. This submerged culture methodology has significant limitations when growing phototrophic organisms as the principle energy source is derived from photons. Photonic energy is sensitive to a line of sight pathway to photoreceptors maintained within phototrophic organisms. Unfortunately, high cell densities in liquid culture result in organism self shading. As a consequence, the vast majority of residence time within the photobioreactor results in non-productive periods for each organism due to the inability to acquire light (photons).

A major issue with current photobioreactors concerns the limits of their deployment to production of value added products is the capital costs associated with photobioreactor construction. The deployment of photobioreactors for commodity chemical production requires a cost generally not to exceed $100,000 per acre. In addition, current bioreactors limit the variables of carbon access to the concentration of CO₂ in the bioreactor atmosphere and the mass transfer/active transport system found within the cultivated organism.

### SUMMARY OF THE INVENTION

One aspect provides a bioreactor suitable for cultivating photosynthetic organisms having an extended mode and a compressed mode.

In some embodiments, a bioreactor includes a feeder base comprising a feeder trough, a collection base comprising a collection trough, a growth fabric coupled to the feeder trough and the collection trough, a barrier layer coupled to the feeder base and the collection base that encases the growth fabric in an airtight environment, and an inlet unit coupled to the feeder trough.

In some embodiments, the bioreactor includes a feeder base comprising a feeder trough, a collection base comprising a collection trough, a growth fabric, and a barrier layer. In some embodiments, the bioreactor has a compressed mode and an extended mode. In some embodiments, the growth fabric is coupled to the feeder trough or the collection trough. In some embodiments, the growth fabric is substantially extended in the extended mode of the bioreactor. In some embodiments, the growth fabric is substantially compressed in the compressed mode of the bioreactor. In some embodiments, the barrier layer is coupled to the feeder base or the collection base to form a substantially airtight environment encasing or substantially encasing the growth fabric.

In some embodiments, the growth fabric is compressed into the collection trough.

In some embodiments, the growth fabric is coupled to a support fabric that extends between the feeder trough and the collection trough inside the barrier layer.

In some embodiments, the bioreactor includes an outlet unit coupled to the collection base or the collection trough.

In some embodiments, the bioreactor includes a gas inlet coupled to the feeder base or the feeder trough and a gas outlet coupled to the collection base or the collection trough.

In some embodiments, the gas inlet includes a filter coupled to the gas inlet.

In some embodiments, the filter is a micron filter.

In some embodiments, the bioreactor includes a condensation outlet, wherein the condensation outlet provides for drainage of moisture condensation from the collection base or the feeder base without diluting or substantially diluting a media solution or an inoculation solution.

In some embodiments, an inoculation solution is injected into the feeder trough by the inoculation solution inlet unit while the bioreactor is compressed.

In some embodiments, the growth fabric is inoculated with a plurality of organisms included in the inoculation solution injected into to feeder trough.

In some embodiments, the bioreactor is extended to an operational mode after the growth fabric is inoculated.

In some embodiments, the used inoculation solution is extracted through an outlet unit on the collection trough.

In some embodiments, the bioreactor includes a sealing unit that seals the barrier layer to the feeder base or the collection base.

In some embodiments, the sealing unit includes at least two gaskets that each engage ridges on both sides of a center portion of the sidewalls of the feeder base or the collection base, a collar unit coupled to the barrier layer that has a central portion that engages the top portion of the sidewall and that comprises at least two extensions configured to engage each gasket, and a clip having a first end that engages a tab on the top surface of the collar and a second end that engages a tab on the lower end of the ridge of the sidewall.

In some embodiments, the clip applies a force that presses the collar unit against the gaskets and ridge to create a hermetic seal.

In some embodiments, the barrier layer is transparent.

In some embodiments, the barrier layer has a portion that is transparent.

In some embodiments, the barrier layer acts a light filter that prevents specific wavelengths of light from entering the bioreactor and that allows other wavelengths of light to enter the bioreactor.

In some embodiments, the sealing unit hermetically seals the barrier layer to the sidewalls of the feeder base or the collection base.

In some embodiments, the bioreactor includes a gas inlet unit on the feeder base or the feeder trough.

In some embodiments, the bioreactor includes a gas outlet unit on the collection base or the collection trough.

In some embodiments, the gas inlet unit includes a filter.

In some embodiments, the feeder base, feeder trough, collection base, collection trough and barrier layer are sized to accommodate a plurality of growth fabrics.

In some embodiments, each of the growth fabrics shares one common inlet port.

Another aspect provides a method of cultivating a photosynthetic organism in a bioreactor described herein. In some embodiments, the method includes creating a substantially airtight or airtight environment by sealing a growth fabric between a feeder trough and collection trough using a barrier layer, compressing the growth fabric into the collection trough by moving the feeder trough towards the collection trough, injecting an inoculation solution into the collection trough by an inlet unit on the feeder trough, submersing the compressed or partially compressed growth fabric in the inoculation solution in the collection trough, and separating the feeder trough from the collection trough such that the growth fabric is fully extended.

In some embodiments, the growth fabric is coupled to a support fabric that extends between the feeder trough and the collection trough.

In some embodiments, an outlet unit is coupled to the collection trough that allows unused inoculation solution to exit the collection trough.

In some embodiments, the method includes the steps of injecting a gas into the bioreactor by a gas inlet coupled to a feeder base or the feeder trough and exhausting excess gas from the bioreactor by a gas outlet coupled to a collection base or the collection trough.

In some embodiments, the gas inlet includes a filter coupled to the gas inlet.

In some embodiments, the filter is a micron filter.

In some embodiments, the growth fabric is inoculated with a plurality of organisms included in the inoculation solution injected into to feeder trough.

In some embodiments, the method includes the step of extracting the unused inoculation solution through an outlet unit on the collection trough.

In some embodiments, the method includes the step of sealing the barrier layer to the feeder base or collection base by a sealing unit.

In some embodiments, the sealing unit includes at least two gaskets that each engage ridges on both sides of a center portion of the sidewalls of the feeder base or the collection base, a collar unit coupled to the barrier layer that has a central portion that engages the top portion of the sidewall and that includes at least two extensions configured to engage each gasket, and a clip having a first end that engages a tab on the top surface of the collar and a second end that engages a tab on the lower end of the ridge of the sidewall.

In some embodiments, the clip applies a force that presses the collar unit against the gaskets and ridge to create a hermetic seal.

In some embodiments, the barrier layer is transparent.

In some embodiments, the barrier layer has a portion that is transparent.

In some embodiments, the barrier layer acts a filter that prevents specific wavelengths of light to enter the bioreactor and that allows other wavelengths of light to enter the bioreactor.

In some embodiments, the sealing unit hermetically seals the barrier layer to the sidewalls of a feeder base comprising the feeder trough ora collection base comprising the collection trough.

In some embodiments, the method includes the step of introducing a gas into the bioreactor by a gas inlet unitfeeder trough.

In some embodiments, the method includes the step of extracting gas from the bioreactor by a gas outlet unit.

In some embodiments, the gas inlet unit includes filter.

In some embodiments, the feeder trough and collection trough are sized to accommodate a plurality of growth fabrics.

In some embodiments, each of the growth fabrics shares one common inlet port.

In some embodiments, the bioreactor includes a frame; a media inlet unit; a media outlet unit; a media delivery tube comprising an intermittent or continuous slit in a substantially lengthwise direction; a multi-layer composite surface for growth and support of microorganisms, the surface comprising a media fabric, a transition layer, and growth fabric; and a barrier layer. In some embodiments, the bioreactor has a compressed mode and an extended mode; the transition layer is sandwiched between and attached to the media fabric and the growth fabric; the media inlet unit, the media delivery tube, and the media outlet unit are fluidly connected; the media fabric is coupled directly to the media delivery tube; the media delivery tube and multi-layer composite surface are supported by the frame; the growth fabric is substantially extended in the extended mode of the bioreactor; the growth fabric is substantially compressed in the compressed mode of the bioreactor; or the barrier layer forms a substantially airtight environment encasing or substantially encasing the growth fabric.

In some embodiments, the composite surface or the growth fabric comprises a three dimensional patterned geometry having increased surface area as compared to a flat surface.

In some embodiments, the media delivery tube comprises a slit, the slit having a first slit face and a second slit face, the first slit face comprising a plurality of closure prongs, and the second slit face comprising a plurality of receiving holes complementary to the closure prongs, such that the closure prongs can pierce at least a portion of the media fabric and be secured in the receiving holes so as to substantially secure the media fabric to the media delivery tube.

In some embodiments, bioreactor includes one or more of a feeder tube, inlet connection valve, an outlet tube, and an outlet connection valve, wherein the media inlet unit, the feeder tube, the inlet connection valve, the media delivery tube, the outlet connection valve, the outlet tube, and the media outlet unit (where present) are fluidly connected.

In some embodiments, the bioreactor includes a rail seat and a support rail, wherein the frame comprises the rail seat, the support rail interfaces with the rail seat, and the support rails interfaces with the media delivery tube.

In some embodiments, the bioreactor includes a support pin, wherein the support pin releasably connects the support rail and the rail seat or the media delivery tube and the support rail.

In some embodiments, the media delivery tube follows a non-linear path through the frame of the bioreactor forming a series of substantially parallel multi-layer composite surfaces or a plurality of interconnected media delivery tubes comprise a non-linear path through the frame of the bioreactor forming a series of substantially parallel multi-layer composite surfaces.

Other systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the accompanying claims.

### BRIEF DESCRIPTION OF THE DRAWINGS

The accompanying drawings, that are incorporated in and constitute a part of this specification, illustrate an implementation of the present invention and, together with the description, serve to explain the advantages and principles of the invention. Those of skill in the art will understand that the drawings, described below, are for illustrative purposes only. The drawings are not intended to limit the scope of the present teachings in any way. In the drawings:

FIG. 1. depicts a front view of one embodiment of a photobioreactor 100.

FIG. 2. depicts a side view of one embodiment of a photobioreactor 100.

FIG. 3A. depicts one embodiment of a photobioreactor 100 in a compact position.

FIG. 3B depicts a side view of one embodiment of the photobioreator 100 in a compact mode.

FIG. 3C depicts a side view of one embodiment of the photobioreactor 100 in a spooled mode.

FIG. 4 depicts one embodiment of a reusable seal that secures a barrier layer to a photobioreactor 100.

FIG. 5 depicts one embodiment of a multi-panel photobioreactor 100.

FIG. 6 depicts a top view of one embodiment of a multi-layer composite surface 120.

FIG. 7 depicts a top view of one embodiment of a patterned composite growth surface.

FIG. 8 depicts a side view of one embodiment of a media delivery tube 130.

FIG. 9 depicts a top down and end view of one embodiment of a continuous culture media feed system 0140 of the photobioreactor 100.

### DETAILED DESCRIPTION OF THE INVENTION

Systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the accompanying claims.

U.S. Pat. Pub. No. 2009/0181434, filed Jan. 5, 2009, is incorporated herein by reference in its entirety.

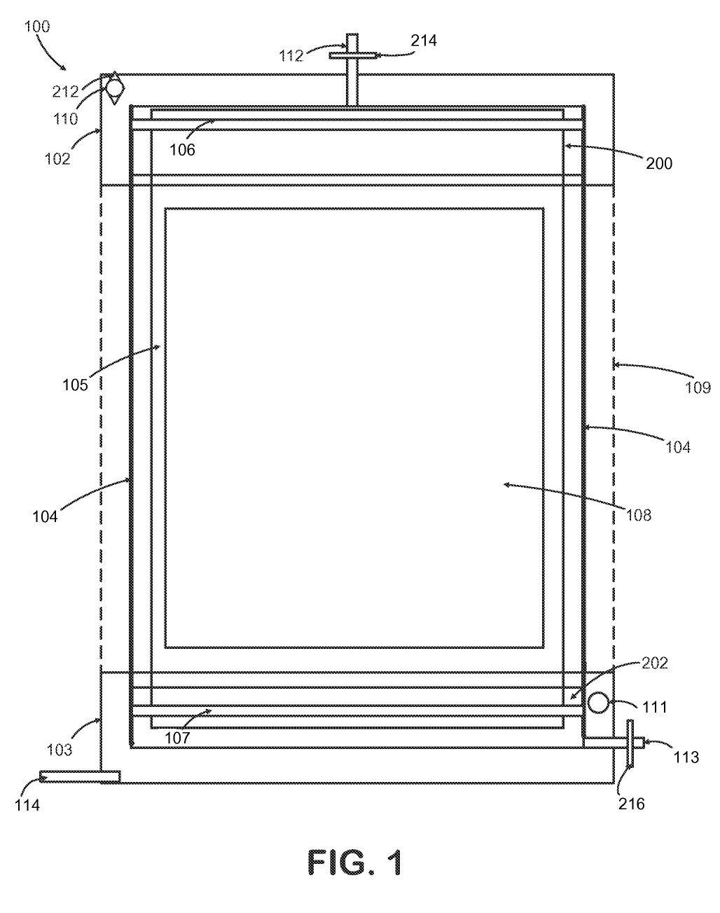

FIG. 1. depicts one embodiment of a photobioreactor 100. Consistent with this embodiment, the photobioreactor 100 includes a feeder base 102, a feeder trough 200, a collection base 103, a collection trough 202, vertical supporting units 104 and a media fabric 105. The feeder trough 200 and collection trough 202 are each coupled to the vertical supporting units 104. In addition, the media fabric 105 is coupled to spooling units 106 and 107, with the spooling unit 106 being coupled to the feeder trough 200 and the spool unit 107 being coupled to the collection trough 202.

A growth fabric 108 is positioned on at least one side of the media fabric 105. The growth fabric 108 can be according to any solid phase cultivation support material or configuration as described in U.S. Pat. Pub. No. 2009/0181434, filed Jan. 5, 2009, which is incorporated herein by reference in its entirety. In some embodiments, the growth fabric 108 and the media fabric 105 are encompassed within the same structure.

A barrier layer 109 is coupled to the feeder base 102 and collection base 103 such that the barrier layer 109 creates a sealed environment for the growth fabric 108, the media fabric 105 and the vertical supporting units 104. The barrier layer 109 can form, for example, an airtight or a substantially airtight environment by way of coupling to the feeder base 102 or collection base 103. In one embodiment, a gas inlet 110 is positioned on the feeder base 102 and a gas outlet 111 is positioned on the collection base 103.

The barrier layer 109 can be according to any barrier layer material or configuration as described in U.S. Pat. Pub. No. 2009/0181434. The photobioreactor 100 can provide for transmission of actinic radiation, either sunlight or artificial light, to the photosynthetic microorganisms. But the barrier layer 109 need not necessarily be transparent to light. Some embodiments can comprise a cultivation support enclosed within a non-transparent protective barrier if a sufficient light source for the growth of photosynthetic microorganisms is provided within. It may be desirable, simpler, more economical, and the like to provide a transparent barrier to utilize sunlight, for instance, as a light source.

Media can be introduced to the feeder trough 200 by a media inlet unit 112 coupled to the feeder trough 200. In some embodiments, media inlet unit 112 can be suspended above the feeder trough 200 without necessarily being fixed or coupled to the feeder trough 200. In some embodiments, media inlet unit 112 can be coupled to the feeder trough 200. Media can be removed from the collection trough 202 by, for example, a media outlet unit 113. Culture media for photosynthetic organisms and use thereof is well known in the art. Except as otherwise noted herein, therefore, culture media can be in accordance with such known formulations and processes. Media can be according to any nutrient or fertilizer solution known in the art for use with the cultured photosynthetic organism. Media supplied to the growth fabric 108 via the feeder trough 200 or through another means can include sufficient water to maintain adequate or optimal moisture, on the growth surface or the surrounding atmosphere, necessary for the cultured photosynthetic organism.

In some embodiments, a condensation outlet unit 114 can be positioned on or near the collection base 103 to allow excess liquid to exit the collection base 103 or the photobioreactor 100. In some embodiments, for example in horizontal configurations, a condensation outlet unit 114 can be positioned on or near the feeder base 103 to allow excess liquid to exit the feeder base 103 or the photobioreactor 100. For example, in a non-vertical configuration, e.g., a horizontal or substantially horizontal configuration, a photobioreactor 100 can include a first condensation outlet unit 114 coupled to the collection base and a second condensation unit 114 coupled to the feeder base 102. Providing a condensation outlet unit 114 separate from the collection trough 202 can provide for drainage of condensate from the bioreactor without diluting the cultivation media or the inoculation media. In some embodiments, a condensation outlet unit 114 provides for drainage of substantial amounts of condensate that would otherwise partially or substantially fill the collection base 103 or collection trough 202 thereby potentially interfering with operation of the bioreactor.

In some embodiments, the media inlet unit 112 and media outlet unit 113 are sealed to the feeder trough 200 and collection trough 202 such that no or substantially no contaminants enter the photobioreactor 100 through the media inlet unit 112 or media outlet unit 103. In some embodiments, the condensation outlet unit 114 is sealed to the collection base 103 such that no or substantially no contaminants enter the photobioreactor 100 through the media inlet unit 112 or media outlet unit 103. The various inlets and outlets, such as media inlet 112, media outlet 113, gas inlet 110, gas outlet 111, and condensation outlet 114 can be located on the sides, front, back, top or bottom of the photobioreactor 100, or components thereof, so long as such placement provides for function as described herein. Placement can be dependent, at least in part, on whether the photobioreactor 100 is in a vertical orientation or a non-vertical orientation, e.g., a horizontal, partially horizontal, or substantially horizontal configuration. In one embodiment, the media inlet unit 112, the media outlet unit 113 or condensation outlet unit 114 are flexible tubes. In another embodiment, the media inlet unit 112, the media outlet unit 113 or condensation outlet unit 114 are rigid tubes.

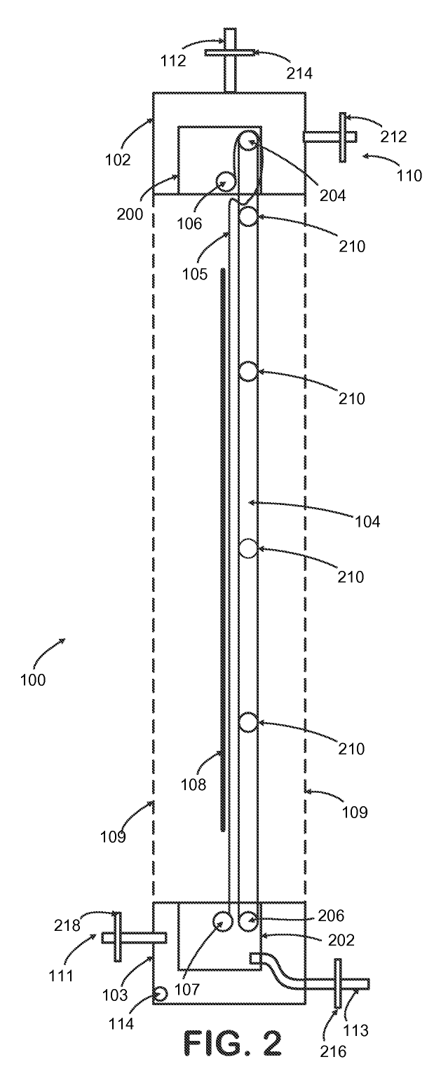

FIG. 2. depicts a side view of the photobioreactor 100. The feeder base 102 and collection base 103 each include a feeder trough 200 and a collection trough 202, respectively. The feeder trough 200 and a collection trough 202 are positioned at opposing ends of each of the feeder base 102 and collection base 103 such that the feeder trough 200 and a collection trough 202 support ends of the spooling units 106 and 107. The spooling units 106 and 107 are coupled to the media fabric 105 such that the rotation of the spooling units 106 and 107 causes the media fabric 105 to spool around one of the spooling units 106 or 107. In one embodiment, the media fabric 105 and growth fabric 108 are spooled around the spooling units 106 and 107 for storage. In some embodiments, the media fabric 105 and growth fabric 108 are incorporated into the same structure, which can be composed of one or more layers.

In some embodiments, the vertical supporting units 104 are coupled to a plurality of supporting rods 210 with the ends of each of the support rods 210 being coupled to the interior sides of the vertical supporting units 104 such that a ladder structure is created. An upper support rod 204 is coupled to the feeder trough 200 or the feeder base 102 and a lower support rod 206 is coupled to the collection trough 202 or the collection base 103. The vertical supporting units 104 are coupled to the support rods 210 and are coupled to the feeder trough 200 and collection trough 202 by the upper support rod 204 and the lower support rod 206 respectively. The vertical supporting units 104 can be made from a flexible material including, but not limited to plastic, paper, cotton or any other flexible material capable of being compressed and extended without breaking In one embodiment, the vertical supporting units 104 are transparent. In another embodiment, only a portion of the vertical supporting units 104 are transparent. In yet another embodiment, the vertical supporting units 104 act as a filter to block transmission of specific wavelengths of light.

The vertical supporting units 104 can be configured to separate the feeder trough 200 from the collection trough 202 in a fully extended mode and to collapse the feeder trough 200 towards the collection trough 202 in a compact mode. The vertical supporting units 104 can be configured to separate the feeder base 102 from the collection base 103 in a fully extended mode and to collapse the feeder base 102 towards the collection base 103 in a compact mode. The support rods 210 provide horizontal support to the media fabric 105 and the growth fabric 108. In one embodiment, the vertical supporting units 104 are canvas straps. In another embodiment, the vertical supporting units 104 are telescoping rods. In yet another embodiment, the vertical supporting units 104 are hydraulic pistons. In another embodiment, an external structure provides support for the photobioreactor 100 and the vertical support units 104 serve to connect the support rods 210 together. In one embodiment, the photobioreactor 100 is arranged in a horizontal position with the collection base 103, or a portion thereof, resting on a support surface. In another embodiment, the photobioreactor 100 is vertically positioned such that the sides of the feeder base 102 and collection base 103, or portions thereof, are in contact with the support surface. Consistent with this embodiment, the support rods 210 can provide vertical support for the growth fabric 108 when the photobioreactor 100 is positioned vertically.

The media fabric 105 is positioned between the vertical support units 104 and the growth fabric 108 and is adhered to the growth fabric 108 such that the growth fabric 108 is vertically positioned between the feeder trough 200 and the collection trough 202. In one embodiment, the media fabric 105 is partially transparent, substantially transparent, or fully transparent. In another embodiment, only a portion of the media fabric 105 is partially transparent, substantially transparent, or fully transparent. In yet another embodiment, the media fabric 105 acts as a filter to block transmission specific wavelengths of light.

The growth fabric 108 is configured to retain organisms for growth in the photobioreactor 100 on the surface of the growth fabric 108. In one embodiment, the growth fabric 108 has a single growth surface. In another embodiment, the growth fabric 108 has two growth surfaces. The growth fabric 108 is a solid phase surface for cultivating cells that avoids solubility and mass transfer issues associated with gaseous CO₂ feedstocks. Organisms are located on the surface of the growth fabric 108 and are exposed directly to the bioreactor atmosphere without significant submersion into a liquid growth medium. Positioning growing cells on the solid surface of the growth fabric 108 eliminates the intermediary mass transfer of gaseous feedstock through a liquid cultivation medium providing greater access to carbon feedstock.

The barrier layer 109 protects the media fabric 108 from contamination or moisture loss while also allowing light to pass through at least a portion of the barrier layer 109. In one embodiment, the barrier layer 109 is made from a material including, but not limited to, polyethylene, acrylic polymers, polyethylene terephthalate, polystyrene, polytetrafluoroethylene, or co-polymers thereof, or combinations thereof. The barrier layer 109 can be selected from materials that are durable and not prone to ripping, tearing, cracking, fraying, shredding, or other such physical damage. The barrier layer 109 can be, for example, a cellulose acetetate or a polyester, e.g., mylar or polyvinylchloride (PVC). The barrier layer 109 material can be selected for its ability to withstand autoclave sterilization or other exposure to temperature extremes. Further, the barrier layer 109 materials can be selected to withstand prolonged exposure to sunlight or other radiation without discoloring or deteriorating.

One of skill in the art will recognize that certain coatings or formulations that resist photooxidation can be particularly useful. In addition, infrared reflecting or absorbing coatings can be selected to reduce or otherwise regulate the buildup of temperature within the photobioreactor of the disclosure. Different configurations of the barrier layer are disclosed in U.S. Pat. Pub. No. 2009/0181434. In some embodiments, the barrier layer can comprise a coating with optical modification properties, such as wave-length filtering (e.g.,. filter out infared radiation or non-photosynthetically active radiation), anti-reflective (e.g., to avoid photon loss and maximize entry of photons into the system), and internally reflectivity (e.g., to maximize photon retention in the system).

The gas inlet 110 is comprised of a tube having one end coupled and sealed to the feeder base 102 with a filter 212 positioned between the feeder base 102 and the end of the tube. The tube is sealed into the feeder base 102 using any conventional methods of sealing including, but not limited to, epoxy sealing, compression sealing or any other method that would create an airtight seal or substantially airtight seal between the tube and the feeder base 102. In one embodiment, the tube is a flexible tube. In another embodiment, the tube is a rigid tube. In another embodiment, the filter 212 is a micron filter. In yet another embodiment, the filter 212 is a 0.1, 0.2, 0.3 or 0.4 micron filter.

The photobioreactor 100 substantially reduces the risk of contamination through a self contained design. An ever present challenge for fermentation is the potential for uncontrolled growth of contaminating organisms that gain access to the growing culture. Such challenge is met by various embodiments of the photobioreactor 100. Often times a contaminating species can out-compete desired organisms because, for example, they have faster growth rates or are not disadvantaged by stresses associated with product biosynthesis. Microbial contamination in conventional bioreactors is most commonly caused by poor sterile technique in preparing the reactor, cross contamination of inoculation cultures or introduction of foreign species during set up and operation of the fermentor. In a conventional bioreactor, open access to the growing culture can occur at poorly sealed fittings and ports or during times when the reactor is opened to introduce necessary components to the fermentation. As described herein, various embodiments of the photobioreactor 100 are a closed system thereby reducing or substantially eliminating the above described issues.

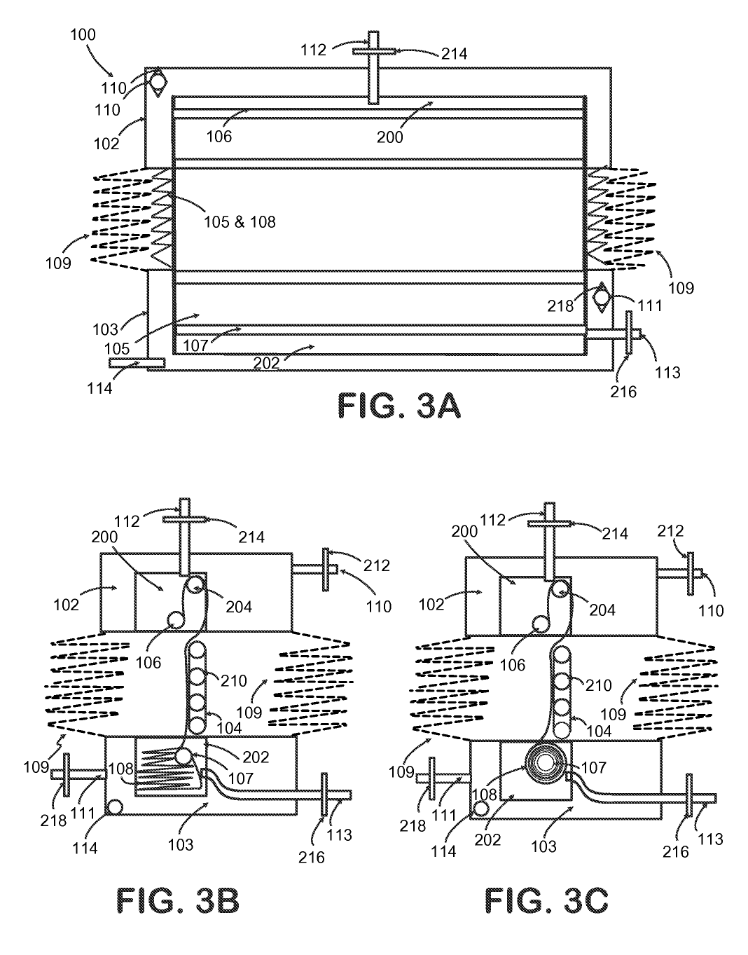

FIG. 3A depicts one embodiment of the photobioreactor 100 in a compact position. The vertical support units 104 compress, or fold, to allow the feeder trough 200 to move towards the collection trough 202. The barrier layer 109 also compresses along with the media fabric 105 and growth fabric 108. In this configuration, the grown fabric 108 is partially contained, substantially contained, or fully contained in the collection trough 202.

A contaminant free preculture solution is introduced into the photobioreactor 100 via the media inlet unit 112 where the media inlet unit 112 includes a filter 214 to prevent contamination of the solution. In another embodiment, the contaminant free preculture solution is introduced to the collection trough 202 through the media outlet unit 113 where the media outlet unit 113 includes a filter 216 to prevent contamination of the solution. In some embodiments, filter 216 is removed before or during an inoculation process. In some embodiments, filter 216 is installed during or after an inoculation process. In one embodiment, the filters 214 and 216 on the media outlet unit 113 and media inlet unit 112 are micron filters. In yet another embodiment, the filters are 0.1, 0.2, 0.3 or 0.4 micron filters. In one embodiment, the preculture solution is injected into the reactor under pressure from a pressure generating device, such as a pump. In another embodiment, the preculture solution is injected into the reactor using gravity. Because the reactor is not opened to inoculate the growth fabric 108, the risk of contamination is greatly reduced.

Once the growth fabric 108 is inoculated by the preculture solution, the spent preculture solution is removed from the photobioreactor 100 through the media outlet unit 113. Solution can be removed from the photobioreactor 100 via the condensation outlet unit 114 in the event of an overflow of the collection trough 202. In one embodiment, the spent preculture solution is removed from the photobioreactor 100 via the media outlet unit 113 under suction from a suction generating device, such as a pump. In another embodiment, the preculture solution is removed from the photobioreactor via the media outlet unit 113 using gravity. In addition, the vertical supporting units 104 are extended such that the feeder trough 200 is separated from the collection trough 202, causing the media fabric 105 to expand. When the growth fabric 108 is partially or fully extended, light from an external source can pass through the barrier layer 109 to support the growth of organisms on the growth fabric 108. In one embodiment, light from the external source passes through the barrier layer 109 and media fabric 105 such that the front and back surfaces of the growth fabric 108 are exposed to light.

Because of the compacting ability, the photobioreactor 100 does not need to be directly opened to inoculate the growth fabric 108 or deploy the photobioreactor 100. As previously described, the photobioreactor 100 is delivered in collapsed configuration where the growth fabric 108 is folded or spooled into the collection trough 202 or feeder trough 200. The folded growth fabric 108 is then inoculated by a dip coating strategy using a contaminant free preculture solution introduced to the collection trough 202. Accordingly, the risk of contaminating the growth fabric 108 is greatly reduced because the the collection trough 202 and the media outlet unit 113 (to control level of media in the collection trough) allows the growth surface to be evenly coated without exposing the photobioreactor 100 interior to potential contamination.

FIG. 3B depicts a side view of one embodiment of the photobioreator 100 in a compact mode where the growth fabric 108 is compressed into the collection trough 202. Consistent with this embodiment, the feeder trough 200 is moved towards the collection trough 202 such that the growth fabric 108 collects in the collection trough 202. During the compacting mode, the barrier layer 109 compacts while maintaining the airtight seal or substantially airtight seal with the feeder base 102 and the collection base 103. In some embodiments, once the growth fabric 108 is contained in the collection trough 202, the preculture solution is introduced into the photobioreactor 100 through the media inlet unit 112 on the feeder trough 200 or the media outlet unit 113 on the collection trough 202. In other embodiments, the preculture solution is introduced into the photobioreactor 100 through the media inlet unit 112 on the feeder trough 200 or the media outlet unit 113 on the collection trough 202 before the growth fabric 108 is contained in the collection trough 202. In other embodiments, the preculture solution is introduced into the photobioreactor 100 through the media inlet unit 112 on the feeder trough 200 or the media outlet unit 113 on the collection trough 202 during the period when the growth fabric 108 is gathering in the collection trough 202.

FIG. 3C depicts a side view of one embodiment of the photobioreactor 100 in a spooled mode. Consistent with this embodiment, the growth fabric 108 or media fabric 105 is spooled around the spooling unit 107. In one embodiment, the spooling unit 107 rotates in a counterclockwise or clockwise motion such that the growth fabric 108 or media fabric 105 spools around spooling unit 107. In another embodiment, the growth fabric 108 or media fabric 105 is spooled around spooling unit 106 in the feeder trough 200. In one embodiment, the spooling units 106 and 107 both include a knob or handle that allows for rotation of the spooling units 106 and 107 from outside the photobioreactor 100. In another embodiment, the spooling units 106 and 107 include a locking unit that prevents the spooling units 106 and 107 from rotating when not in use.

Carbon dioxide is the principle carbon source for metabolism in photosynthetic organisms operating photoautotrophically. Under ambient temperatures, pressures and environmental conditions, CO₂ is present nominally at about 380 ppm as a gas dispersed in the natural atmosphere. This relatively low level of carbon can limit the rate at which photosynthetic organisms uptake the material for growth. In addition, the solubility of carbon dioxide in aqueous solution is approximately 40 mM at room temperature and atmospheric pressure, which can be 1/10^{th} or lower than when compared to carbon feedstocks generally associated with submerged cultures such as glucose, sucrose, or glycerol. Taken together, the available carbon delivered from atmospheric gas flow and the solubility of CO₂ in aqueous solution combined with the mass transfer concerns of partitioning solutes from a gas to a liquid phase can create a low available carbon concentration to maintain the active cultivation of photosynthetic organisms.

Direct exposure of organisms into atmospheric CO₂ can optimize the growth rate based upon carbon feedstock. Access to carbon feedstock can be of increasing importance as the cell density increases. In one embodiment, atmospheric CO₂ is introduced to the photobioreactor 100 by the gas inlet unit 110 and is exhausted through the gas outlet unit 111. The gas inlet unit 110 includes a filter 212 that removes any contaminants in the atmospheric CO₂. In one embodiment, the gas inlet unit 110 acts as a one way valve allowing atmospheric CO₂, or other gas, to move into the photobioreactor 100 without allowing the atmospheric CO₂, or other gas, to move out of the photobioreactor 100. In another embodiment, the CO₂, or other gas, is introduced to the photobioreactor under pressure from a pressure generating device such as, but not limited to, a pump.

The atmospheric CO₂, or other gas, is removed from the photobioreactor 100 through the gas outlet unit 111. The gas outlet unit 110 includes a filter 218 that removes any contaminants in the air stream from the bioreactor or any contaminants from the atmosphere if the gas outlet unit 110 is used as an inlet. Filter 218 can be according to other filters described herein. In one embodiment, the gas outlet unit 111 acts as a one way valve allowing gas to flow out of the photobioreactor 100, but not into the photobioreactor 100. In another embodiment, the gas outlet unit 111 removes atmospheric CO₂, or other gases, from the photobioreactor 100 under suction pressure from a suction pressure generating device such as, but not limited to, a pump.

In one embodiment, additional CO₂, or another gas, is injected into the photobioreactor 100 from an external source. In one embodiment, the additional CO₂ is mixed with the atmospheric CO₂ before entering the photobioreactor 100. In another embodiment, the additional CO₂ is injected into the photobioreactor 100 separate from the atmospheric CO₂.

In one embodiment, a computer system monitors the CO₂ concentration in the photobioreactor 100 and injects or exhausts CO₂ to maintain a constant CO₂ concentration in the photobioreactor. The computer system includes a memory, a processor, a plurality of ports for connecting sensors configured to convert environmental measurements into an electrical signal, a plurality of switches capable of controlling the supply of power to electrical devices such as actuators and signal generators capable generating analog signals. The environmental measurements include, but are not limited to, pressure, temperature, gas concentration, strain, gas flow or any other measurable force or environmental condition.

The computer system executes programs on the processor that monitors forces and environmental conditions in the photobioreactor 100 and that toggle switches or generate analog signals to control devices to control the environment in the photobioreactor 100. As an illustrative example, the computer system measures the amount atmospheric CO₂ flowing into and out of the photobioreactor 100 and sends an analog signal to an actuator coupled to a valve on the gas inlet unit 110 to increase or decrease the amount of atmospheric CO₂ injected into the system to maintain a desired atmospheric CO₂ setpoint in the photobioreactor 100.

Direct exposure of organisms in the photobioreactor 100 to atmospheric CO₂ optimizes the growth rate based upon carbon feedstock availability in the current invention over submerged culture photobioreactors. In addition, photobioreactor 100 allows for better control of CO₂ concentrations available to cultivated cells. The reactor atmosphere can be supplemented with CO₂ gas that provides higher concentrations of feedstock carbon to growing cells. While atmospheric CO₂ and CO₂ have been used to describe gases injected into the photobioreactor, any gas or mixture of gases can be injected into the photobioreactor 100.

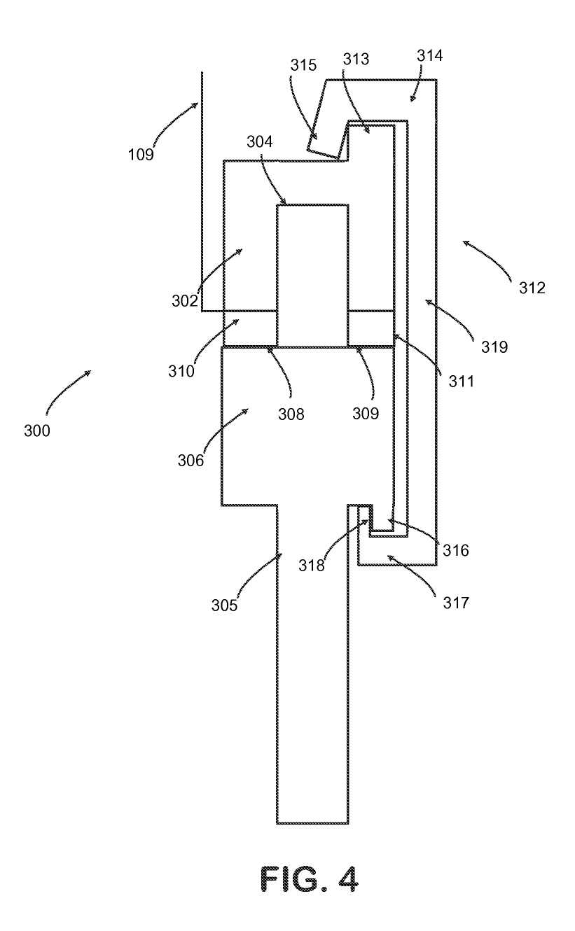

FIG. 4 depicts one embodiment of a reusable seal 300 that secures the barrier layer 109 to the photobioreactor 100. The seal 300 includes a barrier layer collar 302 that is welded to the barrier layer 109. In another embodiment, the barrier layer 109 is secured to the barrier collar 109 by an adhesive. The barrier layer collar 302 is substantially “U” shaped with the center portion 304 of the barrier layer collar 302 configured to engage a sidewall 305 of the feeder base 102 or the collection base 103.

The sidewalls 305 of the feeder base 102 or the collection base 103 include a center portion 306 that has a wider cross section than the other portions of the sidewalls such that ridges 308 and 309 are formed on the side of the center portion 306 closest to the barrier layer 109. Gaskets 310 and 311 are positioned on each of the ridges 308 and 309 such that the sides of the gaskets 310 and 311 are in contact with the ridges 308 and 309 and lower portion of the barrier layer collar 302. The gaskets 310 and 311 are made from a material having memory characteristics including, but not limited to silicon, rubber, latex or any other material having memory characteristics and capable of creating a seal between the sidewall 305 and the barrier layer collar 302.

The center portion 304 of the barrier layer collar 302 engages the unrestrained end of each sidewall to create a hermetic seal with the sidewall 305. The barrier collar 302 is secured to the sidewall 305 by a clip 312 positioned on a side of each sidewall 305 opposite the side holding the barrier layer 109. The clip 312 is substantially “C” shaped with an upper portion of the clip 312 configured to engage a tab 313 on the barrier collar 302. In one embodiment, the tab 313 is substantially square or substantially rectangular in shape. The clip 312 is made from a material having memory characteristics and capable of applying a force on the barrier layer collar 302 to secure the barrier layer collar 302 to the sidewall 305. The clip 312 may be made from materials, including, but not limited to, metal such as steel or titanium, plastic or a metal or plastic covered in a material having memory characteristics such as rubber.

The tab 313 is positioned on one side of the barrier layer collar 302 and extends above the surface of the barrier layer collar 302. The upper portion of the clip 312 includes a horizontal member 314 and a gripping member 315 with the vertical member 319 engaging the top surface of the tab 313 and the gripping member 315 engaging a side surface of the tab 313. In one embodiment, the gripping member 315 is perpendicular in relation to the vertical member 319. In another embodiment, the gripping member 315 is angled towards the tab 313.

The bottom portion of the clip 312 engages a tab 316 on the center portion of the sidewall 305 positioned in line with the tab 313 on the barrier layer collar 302. The bottom portion of the clip 312 includes a horizontal member 317 that engages the top surface of the tab 316 and a vertical member 318 that engages the side of the tab 316 and the lower surface of center portion 306. The clip 312 is configured such that a force is applied to the lower surface of the center portion 306 and an opposite force is applied to the upper surface of the barrier layer collar 302 such that the barrier layer collar 302 is forced downward towards the center portion 306 causing the gaskets 310 and 311 to compress against the center portion creating a seal.

In one embodiment, the clip 312 is configured to remain on the sidewall. In another embodiment, the clip 312 is removable by applying a force in a direction away from the sidewall 305 to the upper portion or lower portion of the clip 312. In one embodiment, the sidewall 305 may have multiple individual clips 312 along the length of the sidewall 305. In another embodiment, the clip 312 is configured to simultaneously engage all of the sidewalls 305 of the feeder base 102 or the collection base 103. In one embodiment, the clip 312 is a compression clip. In another embodiment, the clip 312 includes springs between the vertical members 314 and 316 and the center shaft 318 that force the angled member 315 and the vertical member 317 into the respective barrier layer collar 302 and center portion 306.

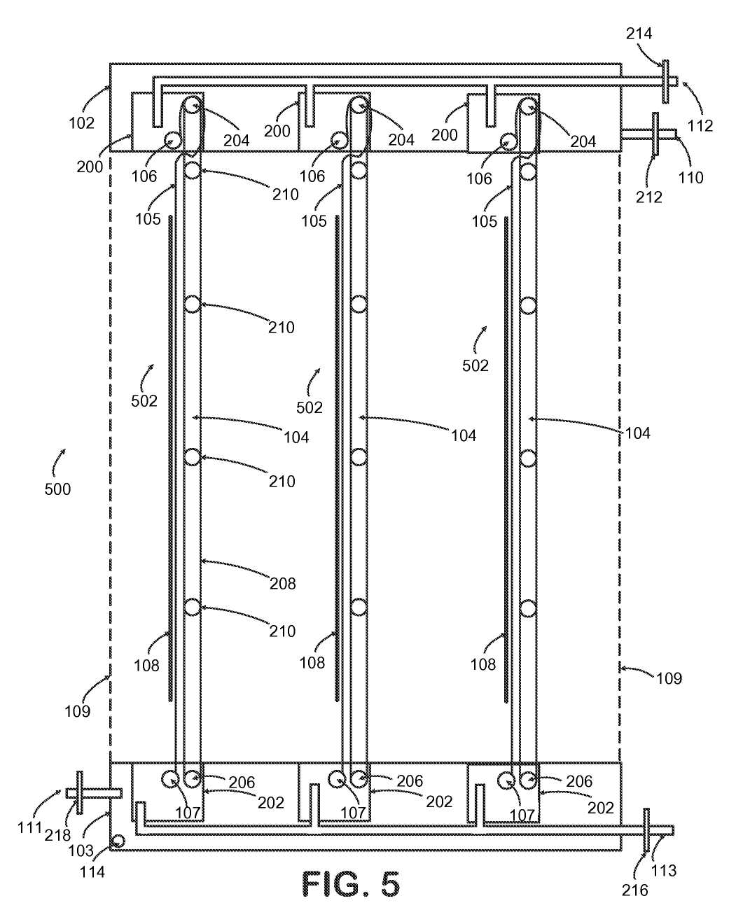

FIG. 5 depicts one embodiment of a multi-panel photobioreactor 500. The multi-panel photobioreactor 500 includes a plurality of growth units 502 with each growth unit 502 including separate support units 104, media fabrics 105, growth fabrics 108 and sealing units 200 and 202. Each of the growth units are coupled to a single feeder trough 200 and a single collection trough 202. The growth units 502 are connected in parallel to a single media inlet unit 112 tube and filter 214 and in parallel to a single media outlet unit 113 tube and filter 216. In another embodiment, each growth unit 502 is individually connected to separate media inlet units 112 and media outlet units 113. In one embodiment, each growth unit 502 is individually connected to separate media inlet units 112 and a common media outlet unit 113. Each growth unit 502 has a separate feeder trough 200 and collection trough 202feeder trough. In some embodiments, each growth unit 502 can share a common feeder trough 200 or a common collection trough 202 sized to accommodate the plurality of growth units 502. In addition, the multi-panel bioreactor includes a single gas inlet unit 110 and a single gas outlet unit 111. In some embodiments, the multi-panel bioreactor includes a plurality of gas inlet units 110 or gas outlet units 111.

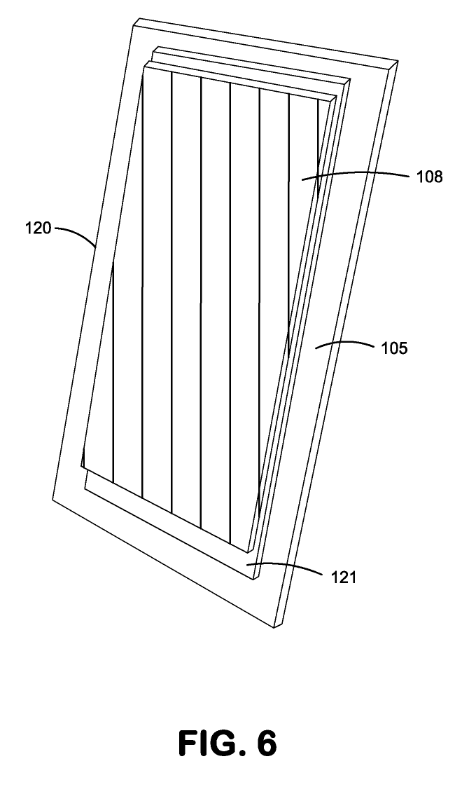

FIG. 6 depicts one embodiment of a multi-layer composite surface 120 for growth and support of microorganisms. The media fabric 105 can be composed of materials as described above. The media fabric 105 can control, wholly or at least in part, the rate at which culture media fluid is transferred through the photobioreactor 100. Attached to the media fabric 105 is the transition layer 121. The transition layer 121 can maintain, wholly or at least in part, contact between the growth fabric 108 and the media fabric 105. Provisional of a transition layer 121 can avoid changes in structural dimension of growth fabric 108 or media fabric 105 as a function of swelling due to, for example, difference between the materials with regard to moisture adsorption or retention. The transition layer 121 can reduce or eliminate delamination of the growth fabric 108 and the media fabric 105. The transition layer 121 can reduce or eliminate accumulation of gas bubbles, such as oxygen bubbles produced by the photosyntehtic microorganisms that can be trapped between the growth fabric 108 and the media fabric 105. The transition layer 121 can maintain intimate contact between the growth fabric 108 and the media fabric 105 and result in consistent fluid transfer between layers.

The transition layer 121 can be composed of a variety of materials, including woven or non-woven fabric, that can be wetted with aqueous solutions and can allow efficient fluid transfer between surfaces. The transition layer 121 can have sufficient thickness, softness or density to allow gases to escape, in whole or in part, while maintaining some or substantial inter-layer contact. The transition layer 121 can enable swelling dependent adjustment between the growth fabric 108 and the media fabric 105 such that some or substantial contact between layers is maintained. The material of the transition layer 121 can include a water resistant adhesive that bonds the growth and media feed layers without partially or substantially impeding fluid transfer. The transition layer 121 can be composed of a hydrophilic polymer, hydrogel or a fabric reinforced hydrophilic polymer or hydrogel. Such materials can provide an additional feature of separating the organisms from the growth fabric 108 and the media fabric 105 or preventing unwanted organism migration or wash off from the photobioreactor 100 by size exclusion.

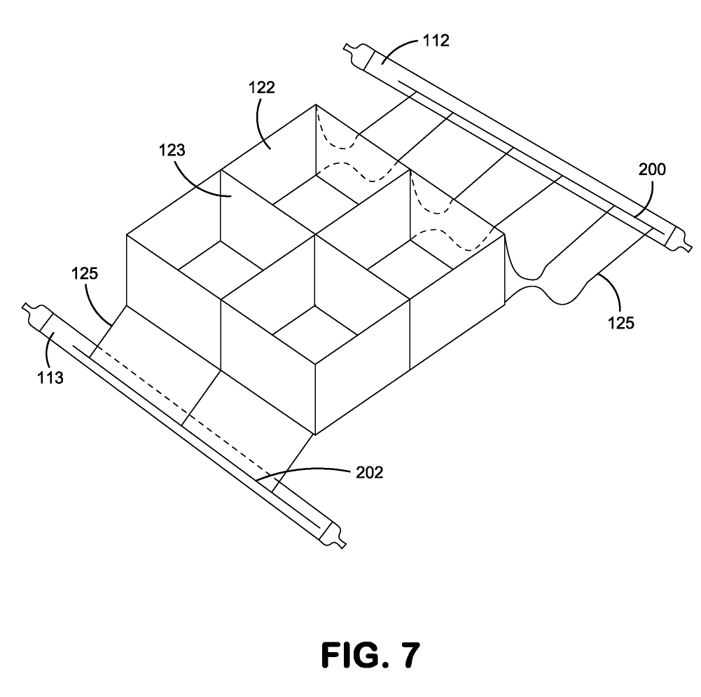

FIG. 7 depicts one embodiment of a patterned composite growth surface. The varied geometry of the patterned composite growth surface can provide increased growing surface area within the photobioreactor 100. The depicted three dimensional checkerboard geometry can be readily assembled and connected to plumbing described above as well as modifications thereto as follows.

A media inlet unit 112 can include tubes 125 or a feeder trough 200 or other system components that can provide for gravity, pressure, or capillary draw mechanism for moving liquid culture media into the media fabric 105. In the depicted embodiment, media inlet unit 112 is a self closing tube that contains the liquid feed within the tube but allows liquid culture media to pass into the media fabric 105 or growth fabric 108.

Vertical wall 122 can be a composite multiple (e.g., comprising growth fabric 108, transition layer 121, and media fabric 105 or growth fabric 108 and media fabric 105) or single surface materials as described previously. Vertical wall 122 can be extended past the crossing components and turned in such a manner that position the media fabric 105 to be positioned into media inlet unit 112. The lower portion of vertical wall 122 can be extended past the last crossing material and part of the media fabric 105 is arranged to provide effluent from the photobioreactor to be collected in the collection trough 202 or media outlet unit 113.

Crossing layer 123 interweaves with vertical wall 122 in such a manner as to allow culture media to wet and permeate crossing layer 123 so as to adequately support organism growth. Crossing layer 123 can be composed of a single or composite multilayered material in similar (but not necessarily the same) manner to vertical wall 122. The surface areas of vertical wall 122 or crossing layer 123 can be increased by adding corrugations or folds along the surface to increase overall growth area. The spacing of the cross pattern can be any size so long as sufficient light can access the most or all of the surface. The overall geometry can optimize height relative to cross pattern spacing so as to achieve sufficient light exposure to growing surfaces. Crossing layer 123 and vertical wall 122 can be held together by any conventional method, such as sewing, fiber entanglement (such as but not limited to hook and loop connectors), thermal welding, or adhesive bonding in so much as fluid transfer through the materials is not partially or substantially adversely impaired.

Media outlet unit 113 provides effluent collection plumbing and can include tubes 125 collection trough 202 such that fluid effluent can be collected and transported out of the photobioreactor 100.

Greater surface area within photobioreactor 100 can provide advantages to photosynthetic cultivation processes. Light entering photobioreactor is often wasted in the sense that photons have a significant probability to strike non-photosynthetic surfaces or be reflected out of the system without being harvested by organisms. Arranging growing organisms in multiple orientations can provide more efficient capture of photon energy. Many photosynthetic organisms are sensitive to direct intense sunlight. In liquid based photobioreactors, organisms reside in a water column of defined pond depth. As the organisms grow within the media they self shade each other and reduce the overall direct light exposure. In a solid state photobioreactor, the growing surface generally affords a thin film of growing organisms on the surface, which prevents substantial self shading as found in liquid based systems. Arranging growing surfaces in high density, complex geometries can provide a mechanism to effectively reduce the overall light exposure to each organism by creating multiple surfaces that afford greater surface area per two dimensional surface thereby reducing the effective photon flux per unit area.

Biomass density can be an important factors driving overall productivity. Photosynthetic organisms are generally difficult to grow to high cell densities in liquid photobioreactors necessary for cost effective bioprocesses. Solid state photobioreactors can position organisms in thin films on high surface area designs, which enable greater biomass accumulation and consequently more efficient, productive photobioreactors. The vertical panel configuration of the photobioreactor 100 can be altered to increase overall growth surface area that can also provide complex surface orientation to distribute light energy more effectively through the photobioreactor.

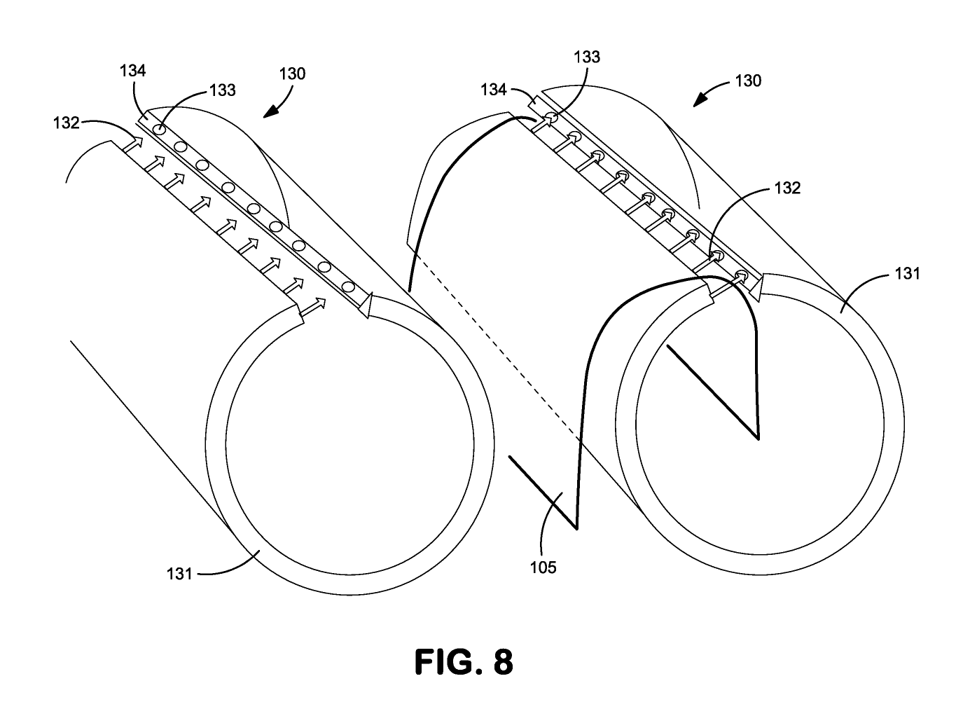

FIG. 8 depicts one embodiment of a media delivery tube 130. The media delivery tube 130 can provide for an active flow of fluid through the photobioreactor 100 under low pressure. While the structure of media delivery tube 130 is described in the following, one of ordinary skill can adapt such description to provide a collection tube. Such a media delivery tube 130 can provide an alternative design to the described feeder trough 200 or collection trough 202 used to deliver (or remove in the case of a collection tube) water and nutrients to (or from) the photobioreactor 100. One of ordinary skill can adapt previously discussed designs including feeder trough 200 or collection trough 202 so as to incorporate media delivery tube 130. The media delivery tube 130 can provide both fluid delivery and serve as an anchor to hold the composite surface 120 (or other surface incorporating one or more of growth fabric 108, transition layer 121, or media fabric 105) within the photobioreactor 100.

The tubing 131 of media delivery tube 130 can be composed of any standard tubing including, but not limited to, flexible plastic such as nylon, tygon, silicone, rubber, or reinforced plastic tubing. Using an additional support material can prevent tubing 131 composed of a flexible materials from collapsing under the weight of the composite surface 120 (or other surface incorporating one or more of growth fabric 108, transition layer 121, or media fabric 105) suspended from tubing 131 or media delivery tube 130. Suitable additional support can include, for example, rigid bars, “L” brackets or secondary rigid tubing composed of acrylic, PVC, polycarbonate, fiberglass, aluminum, stainless steel or other similar material so as to reinforce the tubing 131 or media delivery tube 130.

In the depicted embodiment, media fabric 105 is inserted into media delivery tube 130. Similarly, another surface incorporating one or more of growth fabric 108, transition layer 121, or media fabric 105 could likewise by inserted into media delivery tube 130. Further description below refers to media fabric 105 but one of ordinary skill will understand such discussion also applies to other surfaces incorporating one or more of growth fabric 108, transition layer 121, or media fabric 105. Media fabric 105 can be held in place by the natural recoil force of tubing 131 depending on the tubing thickness or by connectors, such as strings composed of natural or synthetic material. Connectors can be threaded through the media fabric 105 and around the media delivery tube 130 to provide anchoring or pinching the media fabric 105 into the media delivery tube 130. Another form of connector includes closure prongs 132 and receiving holes 133 in receiving bar 134. In this embodiment, media fabric 105 is fed into media delivery tube 130 and impaled on closure prongs 132. Receiving bar 134 includes receiving holes 133 that are complementary to closure prongs 132 with a diameter such that closure prongs 132 are retained once inserted. Closure prongs 132 are inserted into complementary receiving holes 133. Retention of closure prongs 132 can be aided by, for example, barbed tips that click into place when inserted into receiving bar 134. Closure prongs 132 can be singly or multiply barbed. Closure prongs 132 or receiving bar 134 can be composed of any suitable material, such as plastic. Closure prongs 132 or receiving bar 134 can be fabricated into tubing 131 directly or installed separately and held in place by, for example, a tension “U” bracket. Tension closure of closure prongs 132 and receiving bar 134 can be sufficient to provide a seal with media fabric 105 such that culture media can be drawn by capillary action through media fabric 105 without allowing free flowing culture media to substantially leak from media delivery tube 130.

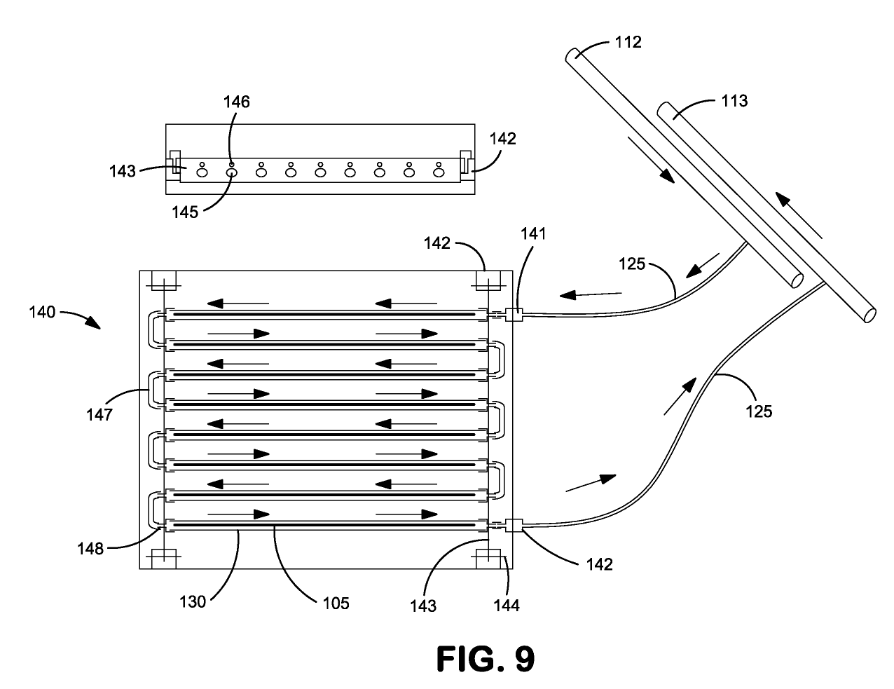

FIG. 9 depicts a top down and end view of one embodiment of a continuous culture media feed system 0140 of the photobioreactor 100. The continuous culture media feed system 0140 can continuously move culture media through the process train supplying multiple photobioreactors 100 in parallel. Such an approach can overcome challenges associated with controlling the flow of liquid and the levels of culture media. A continuous culture media feed system 0140 incorporating a media delivery tube 130 can allow for a single or multiple growth surface photobioreactor 100 that can be fed continuously with reduced concern for managing liquid levels in the feed system. This depiction of a multi-panel design demonstrates the flexibility of the system and is not intended to be limiting with respect to geometry, or quantity of surfaces within the photobioreactor 100.

Media inlet unit 112 and media outlet unit 113 provide a plumbing manifold for supply or collection of culture media. The culture media is maintained in a continuous circuit branched from the main manifold through tubes 125. Tubes 125 can be of any size sufficient to provide enough liquid to maintain growth and productivity of organisms within the photobioreactor 100. Tubes 125 can have a flow control capability to regulate the amount and rate of culture media moving through the photobioreactor 100.

The flow control or pressure within the photobioreactor 100 can be managed by inlet connection valve 141 and outlet connection valve 142. Inlet connection valve 0141 or outlet connection valve 042 can be composed of any suitable material, such as plastic or metal, and represent the connection to the photobioreactor 100 with the rest of the process train. Inlet connection valve 0141 or outlet connection valve 042 can be of any design that allows for the photobioreactor 100 to be attached to the process train by, for example, a sanitary connection or a sanitary push to click connection. Inlet connection valve 0141 or outlet connection valve 042 can work independently or in combination to regulate flow in the system which may be accomplished by, for example, needle, stopcock, butterfly, diaphragm, or iris diaphragm valves. For example, a diaphragm or iris diaphragm valve can regulate flow in the system.

The culture media feed plumbing (e.g., tubes 125, inlet connection valve 0141 or outlet connection valve 042) can be supported within the photobioreactor 100 by rail seat 142 and support rail 143. Rail seat 142 can be composed of any suitable material, such as plastic or metal. Rail seat 142 can include a slit or similar structure in or on which support rail 143 is seated. Rail seat 142 can be a separate piece fixed to the photobioreactor frame by adhesive or a fastener, such as a screw. Rail seat 142 can be consolidated with an injection molded rigid top frame of the photobioreactor 100. Support rail 143 can be seated into rail seat 142 and held in place with locking pin 144. Locking pin 144 can be any suitable design known to those skilled in the art. Support rail 143 can be composed of any suitable material, such as plastic or metal. For example, support rail 143 can be composed of rigid plastic. As another example, support rail 143 can be composed aluminum so as to provide increased support of the weight load of the continuous culture media feed system 0140 and the composite surface 120 (or other surface incorporating one or more of growth fabric 108, transition layer 121, or media fabric 105) within the photobioreactor 100. Support rail 143 can have a support hole 145 (e.g., machined into the support rail 143) to hold the feed plumbing. Support rail 143 can have a second hole 146 (e.g., machined into the support rail 143) for a support pin associated with the feed plumbing to prevent the tubing from rotating under the weight load of the composite surface 120. Alternatively support rail 143 can have a square notch which seats the culture media feed plumbing having a complementary notch to prevent rotation of the feed plumbing under the weight load of the composite growth material. Media delivery tube 130 can be as described above.

For multi-panel photobioreactor 100 designs, media delivery tube 130 can be a continuous flexible tube reinforced as described above or can be a combination of individual reinforced media delivery tubes 130 connected in series by connector 147 (e.g., flexible plastic U shaped tubes) interfacing with media delivery tube 130 via, for example, a friction fit, or a barbed connector 148 (e.g., plastic barbed connector). In a multi-panel design, media delivery tube 130 can makes its way through the photobioreactor 100 at low pressure in, for example, a serpentine pattern and exit the photobioreactor via tube 125 for return to media outlet unit 113.

The photobioreactor 100 provides a low cost effective device to cultivate organisms that incorporates a simplified inoculation mechanism that promotes the growth of organisms by maintaining a contaminant free environment in the photobioreactor. In addition, the photobioreactor can be reused for multiple growth cycles and is easy to clean and redeploy. The device is constructed from robust components that are lightweight, translucent and resistant to environmental conditions. The modular nature of the device allows for rapid servicing without disrupting the entire operational train, thereby maximizing production time.

The photobioreactor 100 can be suspended or conveyed as described in U.S. Pat. Pub. No. 2009/0181434. For example, the photobioreactor can be part of a system including a conveyance system that moves the bioreactor 100 so as to optimize position of the growth fabric 108 for receiving light. As another example, the photobioreactor can be part of a system including a plurality of growth fabrics 108 radiating outward from a central point, or a plurality of photobioreactors radiating outward from a central point. As another example, the photobioreactor can be part of a system including a conveyance system that moves a plurality of growth fabrics 108 around the central point so as to optimize position of one or more growth fabric 108 for receiving light.

A photobioreactor 100, as described herein, can be used for cultivating photosynthetic organisms. Photosynthetic organisms that can be grown in the solid phase photobioreactor include, but are not limited to, a naturally photosynthetic microorganism, such as a higher plant, an algae, acyanobacterium, or an engineered photosynthetic microorganism, such as an artificially photosynthetic bacterium. Exemplary organisms that are either naturally photosynthetic or can be engineered to be photosynthetic include, but are not limited to, bacteria; fungi; archaea; protists; microscopic plants, such as a green algae; and animals such as plankton, planarian, and amoeba. Examples of naturally occurring photosynthetic microorganisms that can be grown in the bioreactor include, but are not limited to, Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechoccus sp., Synechocystis sp., or Tolypothrix. Photosynthetic organisms that can be cultivated in a photobioreactor 100 include photosynthetic organisms described in U.S. Pat. Pub. No. 2009/0181434. Density of photosynthetic organisms cultivated in a photobioreactor 100, including grams of dry biomass per liter equivalent, can be as described in U.S. Pat. Pub. No. 2009/0181434. In some embodiments, a higher plant, such as an orchid, can be grown in the photobioreactor, for example, from a tissue culture sample.

Culture and growth of photosynthetic microorganisms are known to those of ordinary skill in the art. Except as otherwise noted herein, therefore, culture and growth of photosynthetic microorganisms can be carried out in accordance with such known processes. One of ordinary can adapt methods of cultivation of photosynthetic organisms described in U.S. Pat. Pub. No. 2009/0181434 to a photobioreactor 100 described herein. A photobioreactor 100 can be used to cultivate a transgenic cyanobacteria engineered to accumulate a sugar, such as a disaccharide, as described in U.S. Pat. Pub. No. 2009/0181434. Accumulated sugar from a transgenic cyanobacteria or other photosynthetic organism can be harvested or collected from media outlet unit 113, collection trough 202, media fabric 105, or a combination thereof, directly from the media or by harvesting the photosynthetic organisms and isolating the sugar therefrom. In some embodiments, a volatile product (e.g., ethanol) can be harvested or collected from any of the above or the condensation outlet unit 114.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

What is claimed is:

1. A compressible bioreactor for cultivating photosynthetic microorganisms, the bioreactor comprising:
a feeder base comprising a feeder trough;
a collection base comprising a collection trough;
a growth fabric; and
a barrier layer,
wherein,
the bioreactor has a compressed mode and an extended mode;
the growth fabric is coupled directly or indirectly to the feeder trough or the collection trough;
the growth fabric is substantially extended in the extended mode of the bioreactor;
the growth fabric is substantially compressed in the compressed mode of the bioreactor; and
the barrier layer is coupled to the feeder base or the collection base to form a substantially airtight environment encasing or substantially encasing the growth fabric.

2. The bioreactor of claim 1 comprising an inlet unit coupled to the feeder trough or the feeder base.

3. The bioreactor of claim 1 comprising a support fabric, wherein the growth fabric is coupled to the support fabric extending between the feeder trough and the collection trough inside the barrier layer.

4. The bioreactor of claim 1 comprising an outlet unit coupled to the collection trough or the collection base.

5. The bioreactor of claim 1 comprising a gas inlet coupled to the feeder base or the feeder trough or a gas outlet coupled to the collection base or the collection trough.

6. The bioreactor of claim 5 wherein the gas inlet includes a filter coupled to the gas inlet.

7. The bioreactor of claim 1 comprising a condensation outlet, wherein the condensation outlet provides for drainage of moisture condensation from the collection base or the feeder base without diluting or substantially diluting a media solution or an inoculation solution.

8. The bioreactor of claim 2 wherein the bioreactor comprises an inoculation mode of operation where an inoculation solution is introduced into the feeder trough by the inlet unit before, during or after the bioreactor is compressed.

9. The bioreactor of claim 8 wherein the inoculation solution comprises a plurality of photosynthetic microorganisms and the growth fabric is inoculated with the plurality of photosynthetic microorganisms during the inoculation mode of operation.

10. The bioreactor of claim 9, wherein in an operational mode after the growth fabric is inoculated, the feeder trough and collection trough are separated to fully extend the growth fabric.

11. The bioreactor of claim 8 wherein the inoculation solution is extracted through an outlet unit on the collection trough.

12. The bioreactor of claim 1, comprising a sealing unit that seals the barrier layer to the feeder trough or the collection trough.

13. The bioreactor of claim 12 wherein the sealing unit comprises
at least two gaskets that each engage ridges on both sides of a center portion of the sidewalls of the feeder base or the collection base;
a collar unit coupled to the barrier layer that has a central portion that engages the top portion of the sidewall and that includes at least two extensions configured to engage each gasket; and
a clip having a first end that engages a tab on the top surface of the collar and a second end that engages a tab on the lower end of the ridge of the sidewall.

14. The bioreactor of claim 13 wherein, the clip applies a force that presses the collar unit against the gaskets and ridge to create a hermetic seal.

15. The bioreactor of claim 1, wherein the barrier layer is transparent.

16. The bioreactor of claim 1, wherein the barrier layer has a portion that is transparent.

17. The bioreactor of claim 1, wherein the barrier layer acts a light filter that prevents specific wavelengths of light from entering the bioreactor and that allows other wavelengths of light to enter the bioreactor.

18. The bioreactor of claim 13 wherein the sealing unit hermetically seals the barrier layer to the sidewalls of the feeder base or the collection base.

19. The bioreactor of claim 1 comprising a gas inlet unit on the feeder base or the feeder trough.

20. The bioreactor of claim 1 comprising a gas outlet unit on the collection base or the collection trough.

21. The bioreactor of claim 19 wherein the gas inlet unit includes filter.

22. The bioreactor of claim 1 wherein the feeder base, feeder trough, collection base, collection trough and barrier layer are sized to accommodate a plurality of growth fabrics.

23. The bioreactor of claim 22 wherein each of the growth fabrics shares one common inlet port.

24. A method of cultivating an organism in a bioreactor comprising the steps of:
creating an airtight environment by sealing a growth fabric between a feeder trough and collection trough using a barrier layer;
compressing the growth fabric into the collection trough by moving the feed trough towards the collection trough;
injecting an inoculation solution into the collection trough by an inlet unit on the feeder trough;
submersing the compressed growth fabric in the inoculation solution in the collection trough; and
separating the feeder trough from the collection trough such that the growth fabric is fully extended.

25. The method of claim 24 wherein, the growth fabric is coupled to a support fabric that extends between the feeder trough and the collection trough.

26. The method of claim 24 wherein an outlet unit is coupled to the collection trough that allows unused inoculation solution to exit the collection trough.

27. The method of claim 24 comprising the steps of
injecting a gas into the bioreactor by a gas inletfeeder trough; and
exhausting excess gas from the bioreactor by a gas outlet.

28. The method of claim 26 wherein the gas inlet includes a filter coupled to the gas inlet.

29. The method of claim 28 wherein the filter is a micron filter.

30. The method of claim 24 wherein the growth fabric is inoculated with a plurality of organisms included in the inoculation solution injected into the feeder trough.

31. The method of claim 24 comprising the step of extracting the unused inoculation solution through an outlet unit on the collection trough.

32. The method of claim 24 comprising the step of sealing the barrier layer to a feeder base comprising the feeder trough and a collection base comprising the collection trough by a sealing unit.

33. The method of claim 33 wherein the sealing unit includes
at least two gaskets that each engage ridges on both sides of a center portion of the sidewalls of the feeder base and the collection base;
a collar unit coupled to the barrier layer that has a central portion that engages the top portion of the sidewall and that includes at least two extensions configured to engage each gasket; and
a clip having a first end that engages a tab on the top surface of the collar and a second end that engages a tab on the lower end of the ridge of the sidewall.

34. The method of claim 33 wherein, the clip applies a force that presses the collar unit against the gaskets and ridge to create a hermetic seal.

35. The method of claim 24, wherein the barrier layer is transparent.

36. The method of claim 24, wherein the barrier layer has a portion that is transparent.

37. The method of claim 24, wherein the barrier layer acts a filter that prevents specific wavelengths of light to enter the bioreactor and that allows other wavelengths of light to enter the bioreactor.

38. The method of claim 33 wherein the sealing unit hermetically seals the barrier layer to the sidewalls of the feeder base and the collection base.

39. The method of claim 24 comprising the step of introducing a gas into the bioreactor by a gas inlet unit.

40. The method of claim 40 comprising the step of extracting gas from the bioreactor by a gas outlet unit.

41. The method of claim 39 wherein the gas inlet unit includes filter.

42. The method of claim 24 wherein the feeder trough and collection trough are sized to accommodate a plurality of growth fabrics.

43. The method of claim 42 wherein each of the growth fabrics shares one common inlet port.

44. A compressible bioreactor for cultivating photosynthetic microorganisms, the bioreactor comprising:
a frame;
a media inlet unit;
a media outlet unit;
a media delivery tube comprising an intermittent or continuous slit in a substantially lengthwise direction;
a multi-layer composite surface for growth and support of microorganisms, the surface comprising a media fabric, a transition layer, and growth fabric; and
a barrier layer,
wherein,
the bioreactor has a compressed mode and an extended mode;
the transition layer is sandwiched between and attached to the media fabric and the growth fabric;
the media inlet unit, the media delivery tube, and the media outlet unit are fluidly connected;
the media fabric is coupled directly to the media delivery tube;
the media delivery tube and multi-layer composite surface are supported by the frame;
the growth fabric is substantially extended in the extended mode of the bioreactor;
the growth fabric is substantially compressed in the compressed mode of the bioreactor; and
the barrier layer forms a substantially airtight environment encasing or substantially encasing the growth fabric.

45. The bioreactor of claim 44, wherein the composite surface or the growth fabric comprises a three dimensional patterned geometry having increased surface area as compared to a flat surface.

46. The bioreactor of claim 44, wherein the media delivery tube comprises a slit, the slit having a first slit face and a second slit face, the first slit face comprising a plurality of closure prongs, and the second slit face comprising a plurality of receiving holes complementary to the closure prongs, such that the closure prongs can pierce at least a portion of the media fabric and be secured in the receiving holes so as to substantially secure the media fabric to the media delivery tube.

47. The bioreactor of claim 44, comprising a feeder tube, inlet connection valve, an outlet tube, and an outlet connection valve, wherein the media inlet unit, the feeder tube, the inlet connection valve, the media delivery tube, the outlet connection valve, the outlet tube, and the media outlet unit are fluidly connected.

48. The bioreactor of claim 44, comprising a rail seat and a support rail, wherein the frame comprises the rail seat, the support rail interfaces with the rail seat, and the support rails interfaces with the media delivery tube.

49. The bioreactor of claim 48, comprising a support pin, wherein the support pin releasably connects the support rail and the rail seat or the media delivery tube and the support rail.

50. The bioreactor of claim 44, wherein the media delivery tube comprises a non-linear path through the frame of the bioreactor forming a series of substantially parallel multi-layer composite surfaces or a plurality of interconnected media delivery tubes comprise a non-linear path through the frame of the bioreactor forming a series of substantially parallel multi-layer composite surfaces.
